(19) United States
(12) Patent Application Publication
(10) Pub. No.: US 2025/0213559 A1
(43) Pub. Date: Jul. 3, 2025

(54) COMPOSITIONS AND METHODS FOR PREVENTING AND TREATING CYTOKINE RELEASE SYNDROME

(71) Applicant: Alexion Pharmaceuticals, Inc., Boston, MA (US)

(72) Inventors: Pamela B. Conley, Palo Alto, CA (US); Gregory Coffey, San Carlos, CA (US); Matthew Birrell, Lafayette, CA (US)

(73) Assignee: Alexion Pharmaceuticals, Inc., Boston, MA (US)

(21) Appl. No.: 18/850,545

(22) PCT Filed: Mar. 24, 2023
(86) PCT No.: PCT/US2023/016191

§ 371 Date: Sep. 24, 2024

Related U.S. Application Data
(60) Provisional application No. 63/326,100, filed on Mar. 31, 2022.

(51) Int. Cl. A61K 31/506; A61K 45/06

(52) U.S. Cl. CPC A61K 31/506; A61K 45/06

(57) ABSTRACT

Compositions and methods are provided that are useful for treating or preventing cytokine release syndrome, which is a major cause of deaths in infections with virulent viruses such as the coronaviruses and influenza viruses. Therapies such as immunomodulators and CAR-T have also been associated with cytokine release syndrome. The treatment includes the administration of a SYK inhibitor or a pharmaceutically acceptable salt thereof. preferably a dual SYK/JAK inhibitor or a pharmaceutically acceptable salt thereof.

### CROSS REFERENCE TO RELATED APPLICATIONS

This application claims the benefit under 35 U.S.C. § 119 (e) of U.S. Provisional Application Ser. No. 63/326,100, filed March 31. 2022. the content of which is incorporated by reference in its entirety into the present disclosure.

### BACKGROUND

Cytokine release syndrome (CRS) is a form of systemic inflammatory response syndrome that occurs when large numbers of white blood cells are activated and release inflammatory cytokines, which in turn activate even more white blood cells. Severe cases have been called cytokine storms.

It is believed that cytokine storms were responsible for the disproportionate number of healthy young adult deaths during the 1918 influenza pandemic, which killed 50 to 100 million people. Therefore, a healthy immune system may have been a liability rather than an asset. Cytokine storm was also the probable reason for many deaths during the SARS epidemic in 2003, the bird flu H5N1 epidemic, and the 2019-20 coronavirus pandemic, and has been implicated in hantavirus pulmonary syndrome.

Symptoms of CRS include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, and loss of coordination.

CRS occurs when large numbers of white blood cells, including B cells, T cells, natural killer cells, macrophages, dendritic cells, and monocytes are activated and release inflammatory cytokines, which in turn activate vet more white blood cells. This can occur when the immune system is fighting pathogens, as cytokines signal immune cells such as T-cells and macrophages to travel to the site of infection. In addition, cytokines activate those cells, stimulating them to produce more cytokines.

CRS occurs in the context of sepsis. The consequences of sepsis are a result not of the pathogen, but rather the immune response to the pathogen. Tumor necrosis factor (TNF) and interleukin-1β (IL-1β) are identified as major inflammatory cytokines in models of sepsis.

Severe CRS can occur in a number of infectious and non-infectious diseases including graft-versus-host disease (GVHD), acute respiratory distress syndrome (ARDS), sepsis, Ebola, avian influenza, smallpox, and systemic inflammatory response syndrome (SIRS). Hemophagocytic lymphohistiocytosis and Epstein-Barr virus-related hemophagocytic lymphohistiocytosis are caused by extreme elevations in cytokines and can be regarded as one form of severe cytokine release syndrome.

Iatrogenic (i.e., induced inadvertently by medical procedure) causes of CRS are also common. The entire list of iatrogenic causes of cytokine storm is extensive, including pharmacologic (e.g., rituximab therapy), to procedural (e.g., cardiac bypass). The use of chimeric antigen receptor (CAR) T cells in treatments has recently been found to cause CRS as well.

CRS cannot be considered a disease itself, but rather the common end point of different initial insults: infectious, autoimmune/inflammatory, and iatrogenic. Even within those broad categories significant differences exist, making the landscape unlikely to be amenable to a “one-size-fits-all” therapy. Treatment for less severe CRS is supportive, addressing the symptoms like fever, muscle pain, or fatigue. Moderate CRS requires oxygen therapy and giving fluids and antihypotensive agents to raise blood pressure. For moderate to severe CRS, the use of immunosuppressive agents like corticosteroids may be necessary, but judgement must be used to avoid negating the effect of drugs intended to activate the immune system.

With CRS being one of the major causes of death in the recent and current epidemics and immunotherapy patients, there is an urgent need to identify effective treatments.

### SUMMARY

The present disclosure relates to compositions and methods useful for treating or preventing cytokine release syndrome (CRS) in a subject, e.g., a human patient. One embodiment provides a method for treating or preventing cytokine release syndrome in a patient in need thereof, comprising administering to the patient an effective amount of a SYK inhibitor or a pharmaceutical composition comprising the SYK inhibitor. In some embodiments, the SYK inhibitor is a dual SYK/JAK inhibitor. In some embodiments, the SYK inhibitor is cerdulatinib or a pharmaceutically acceptable salt thereof, e.g., a pharmaceutical composition comprising cerdulatinib or a salt thereof and a pharmaceutically acceptable carrier or excipient.

In some embodiments, the patient has a viral infection, caused by a coronavirus (e.g., SARS-COV-2 or SARS-COV), or an influenza virus.

In some embodiments, the patient has been treated with an immunomodulating agent, such as an anti-CD3 antibody, an anti-CD52 antibody, and an anti-CD20 antibody. In some embodiments, the patient has been treated with T-cells modified with chimeric antigen receptors (CAR-T). In some embodiments, the patient has graft-versus-host disease (GVHD), acute respiratory distress syndrome (ARDS), sepsis, Ebola, avian influenza, smallpox, or systemic inflammatory response syndrome (SIRS). In some embodiments, the patient has had a stem cell transplant.

In some embodiments, the present disclosure relates to a SYK inhibitor, e.g., cerdulatinib or a pharmaceutically acceptable salt thereof, for the treatment or prevention of cytokine release syndrome (CRS) in a subject, e.g., CRS in a human patient due to a viral infection, for example, CRS caused by a coronavirus (e.g., SARS-COV-2 or SARS-COV), or an influenza virus.

In some embodiments, the present disclosure relates to use of a SYK inhibitor, e.g., cerdulatinib or a pharmaceutically acceptable salt thereof, for the treatment or prevention of cytokine release syndrome (CRS) in a subject, e.g., CRS in a human patient due to a viral infection, for example, CRS caused by a coronavirus (e.g., SARS-COV-2 or SARS-COV), or an influenza virus.

Numerous other aspects are provided in accordance with these and other aspects of the disclosure. Other features and aspects of the present disclosure will become more fully apparent from the following detailed description and the appended claims.

### DETAILED DESCRIPTION

### 1. Definitions

Unless defined otherwise, all technical and scientific terms used herein have the same meaning as commonly understood by one of ordinary skill in the art to which this disclosure belongs.

As used herein the following terms have the following meanings. Also as used herein and in the appended claims, the singular forms “a”, “an”, and “the” include plural referents unless the context clearly dictates otherwise. Thus, for example, reference to “an agent” includes a plurality of agents.

The term “about” when used before a numerical designation, e.g., temperature, time, amount, and concentration, including range, indicates approximations which may vary by (+) or (−) 10%, 5% or 1%.

As used herein, an “inhibitor” refers to an agent or molecule that inhibits or binds to, partially or totally blocks stimulation or activity, decreases, closes, prevents, delays activation or enzymatic activity, inactivates, desensitizes, or down regulates the activity of a receptor.

The term “administering” refers to oral administration, administration as a suppository, topical contact, intravenous, intraperitoneal, intramuscular, intralesional, intranasal or subcutaneous administration, or the implantation of a slow-release device e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The terms “treat,” “treating,” “treatment.” and grammatical variations thereof as used herein, includes partially or completely delaying, alleviating, mitigating or reducing the intensity of one or more attendant symptoms of a disorder or condition and/or alleviating, mitigating or impeding one or more causes of a disorder or condition. Treatments as described herein may be applied preventively, prophylactically, palliatively or remedially.

The terms “prevent,” “preventing,” “prevention,” and grammatical variations thereof as used herein, refers to a method of partially or completely delaying or precluding the onset or recurrence of a disorder or condition and/or one or more of its attendant symptoms or barring a subject from acquiring or reacquiring a disorder or condition or reducing a subject's risk of acquiring or requiring a disorder or condition or one or more of its attendant symptoms.

As used herein, the term “therapeutically effective” or “effective amount” indicates that a compound or material or amount of the compound or material when administered is sufficient or effective to prevent, alleviate, or ameliorate one or more symptoms of a disease, disorder or medical condition being treated, and/or to prolong the survival of the subject being treated. The therapeutically effective amount will vary depending on the compound, the disease, disorder or condition and its severity and the age, weight, etc., of the mammal to be treated. The dosage can be conveniently administered, e.g., in divided doses up to four times a day or in sustained-release form.

### 2. Treatments

Disclosed herein are compositions and methods useful for treating or preventing cytokine release syndrome (CRS), which is a major cause of deaths in infections with virulent viruses such as the coronaviruses and influenza viruses. Therapies such as immunomodulators and CAR-T have also been associated with cytokine release syndrome. The treatment, in some embodiments, entails the administration of a SYK inhibitor, preferably a dual SYK/JAK inhibitor.

“Cytokine storm syndrome” or “CRS” is a diverse set of conditions represented by a clinical phenotype of systemic inflammation, multi-organ failure, hyperferritinemia, and, if untreated, often death. This clinical constellation is caused by the elaboration of extreme amounts of inflammatory mediators resulting from unchecked feedforward immune activation and amplification.

The mechanisms of CRS may be diverse. Interferon gamma (IFNγ) may play an important role in some cases. A familial hemophagocytic lymphohistiocytosis (FHLH) patient, for instance, is deficient in the perforin gene. IFNγ release is stimulated when the CD8+T cells recognize an infected cell but are unable to kill the infected cell due to the perforin deficiency.

Among rheumatic diseases (e.g., systemic juvenile idiopathic arthritis (JIA) and adult-onset Still's disease (AOSD)), a form of CRS, macrophage activation syndrome (MAS), is developed due to dysregulation of IL-1β, IL-6 and IL-18.

Patients receiving an immunomodulating therapy (e.g., an anti-CD3 antibody, an anti-CD52 antibody, an anti-CD20 antibody, or CAR-T), on the other hand, have even more complicated mechanisms, likely involving dysfunctional B cells, macrophages, or IL-6.

The mechanistic roles played by viruses in the manifestation of CRS is not precisely characterized. The cause of COVID-19 is SARS-COV-2, a strain of coronavirus. It has been suggested that SARS-COV-2 enters cells through angiotensin-converting enzyme 2 (ACE2). Therefore, lung tissue has become the main invasion target of the SARS-COV-2 virus as the lung expresses high levels of ACE2. After the virus enters the lungs, tissue-associated macrophages initially respond to virus via a class of receptors known as pattern-recognition receptors. This includes the C-type lectin receptor Mincle, which is critically involved in initiation the immune reaction to SARS-COV-2. Mincle activation requires SYK kinase function. Once macrophages are activated via the Mincle/SYK pathway, a variety of cytokines are released, resulting in the infiltration and activation of multiple other immune cells. These immune cells attack the lung tissue indiscriminately, leading to respiratory distress. Research shows that patients with severe COVID 19 had significantly higher levels of plasma pro-inflammatory factors (IL2, IL7, IL-10, GSCF, IP-10, MCP-1, MIPIA, TNF-α) than mild patients.

Accumulating studies indicate that the CRS caused by SARS is mainly related to IL-1β,IL-6, IL12A, IFN-γ, IP10 and MCP1, and that caused by MERS is mainly related to IFNγ, TNFα, IL15 and IL17A.

Inhibition of these dysregulated cytokines, however, has shown mixed results. Inhibition of the spleen tyrosine kinase (SYK) may be effective in ameliorating CRS by preventing the initial viral-mediated activation of macrophages via Mincle engagement. Preferably, the SYK inhibitor is dual SYK/JAK inhibitor, such as cerdulatinib or a pharmaceutically acceptable salt thereof.

In one embodiment, therefore, the present disclosure provides a method for treating cytokine release syndrome in a patient in need thereof. In another embodiment, a method is provided for preventing cytokine release syndrome in a patient in need thereof. In some embodiments, the method entails administering to the patient an effective amount of a SYK inhibitor or a pharmaceutically acceptable salt thereof. In some embodiments, the method entails administering to the patient an effective amount of a JAK inhibitor or a pharmaceutically acceptable salt thereof. In some embodiments, the method entails administering to the patient an effective amount of a dual SYK/JAK inhibitor or a pharmaceutically acceptable salt thereof.

Spleen tyrosine kinase (SYK) is a cytosolic non-receptor protein tyrosine kinase (PTK) and is mainly expressed in hematopoietic cells. SYK was recognized as a critical element in the B-cell receptor signaling pathway, and also a key component in signal transduction from other immune receptors like Fc receptors, integrins, and C-type lectin receptors such as Mincle. Non-limiting examples of SYK inhibitors include fostamatinib (previously known as R788), entospletinib (GS-9973), cerdulatinib, and TAK-659 or their pharmaceutically acceptable salt(s).

A Janus kinase (JAK) inhibitor can inhibit the activity of one or more of the Janus kinase family of enzymes (JAK1, JAK2, JAK3, TYK2), thereby interfering with the JAK-STAT signaling pathway. In some embodiments, the JAK inhibitor is a JAK3 inhibitor or a pharmaceutically acceptable salt thereof. In some embodiments, the JAK inhibitor is a JAK2 inhibitor or a pharmaceutically acceptable salt thereof. In some embodiments, the JAK inhibitor is a JAK1/JAK2 inhibitor or a pharmaceutically acceptable salt thereof. Non-limiting examples of the JAK inhibitor include ruxolitinib, fedratinib, pacritinib, and cerdulatinib, or their pharmaceutically acceptable salt(s).

In some embodiments, the SYK or JAK inhibitor is cerdulatinib or a pharmaceutically acceptable salt thereof. Cerdulatinib is a small molecule, ATP-competitive, reversible inhibitor of both SYK and JAK family members and is described in U.S. Pat. No. 8,138,339 and U.S. Pat. No. 8,501,944. Cerdulatinib has a chemical name of 4-(cyclopropylamino)-2-((4-(4-(ethylsulfonyl)piperazin-1-yl)phenyl)amino)pyrimidine-5-carboxamide, and the structure of formula I or a pharmaceutically acceptable salt thereof:

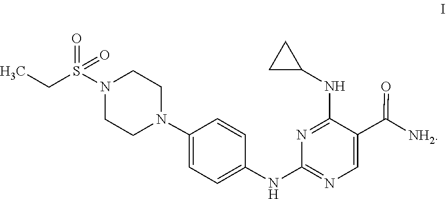 [SMILES: CCS(=O)(=O)N1CCN(c2ccc(Nc3ncc(C(N)=O)c(NC4CC4)n3)cc2)CC1]

In embodiments, the disclosure relates to the hydrochloride salt of cerdulatinib, along with pharmaceutical compositions comprising the same and a pharmaceutically acceptable carrier or excipient.

The present technology is useful for treating or preventing CRS in various types of patients. In one embodiment, the patient has a viral infection. The viral infection, in one embodiment, is caused by a coronavirus including, without limitation, SARS-COV-2 or SARS-COV. SARS-COV-2 has close genetic similarity to bat coronaviruses, from which it likely originated. From a taxonomic perspective, SARS-COV-2 is classified as a strain of the species severe acute respiratory syndrome-related coronavirus (SARSr-COV). In some embodiments, the patient is affected with coronavirus disease 2019 (COVID19).

In some embodiments, the viral infection is an infection by an influenza virus such as, without limitation, the H5N1 influenza A virus or the H7N9 influenza A virus.

In some embodiments, the patient has been treated with an immunomodulating agent. Examples, without limitation, include inhibitors of CD3, CD52, and CD20 (e.g., anti-CD3 antibody Muromonab-CD3, anti-CD52 antibody alemtuzumab, and anti-CD20 antibody rituximab).

In some embodiments, the patient has received a CAR-T (T-cells modified with chimeric antigen receptors) treatment. Chimeric antigen receptor T cells (CAR-T cells) are T cells that have been genetically engineered to produce an artificial T-cell receptor for use in immunotherapy. Chimeric antigen receptors (CARs) are receptor proteins that have been engineered to give T cells the new ability to target a specific protein. The receptors are chimeric because they combine both antigen-binding and T-cell activating functions into a single receptor.

The most common issue after treatment with CAR-T cells is CRS. CRS occurs in almost all patients treated with CAR-T cell therapy; in fact, the presence of CRS is a diagnostic marker that indicates the CAR-T cells are working as intended to kill the cancer cells. A higher grade of CRS severity, however, does not correlate with an increased response to the treatment, but rather higher disease burden.

In some embodiments, the patient suffers from a disease or condition selected from the group consisting of familial hemophagocytic lymphohistiocytosis (FHLH), systemic juvenile idiopathic arthritis (JIA), adult-onset Still's disease (AOSD), macrophage activation syndrome (MAS) and sepsis.

### 3. Dosages

The dosage regime of the SYK or JAK inhibitor (e.g., cerdulatinib) can be determined by the condition of patient, such as the severity and/or types of cytokines/lymphocytes activated. The methods disclosed herein can comprise administering to the patient an effective amount of a SYK or JAK inhibitor (e.g., cerdulatinib), or a pharmaceutically acceptable salt thereof, or a pharmaceutical composition. For example, the method can comprise administering the patient a daily dose of, 15, 20 or 30 mg. In some embodiments, the daily dose may be 5 mg, 8 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, or more, or a number or a range between any two of these values. In some embodiments, the method comprises administering the patient a daily dose of, about 10 mg to about 75 mg of cerdulatinib, or a pharmaceutically acceptable salt thereof, and at least one pharmaceutically acceptable excipient or carrier. In some embodiments, the method comprises administering the patient a daily dose of, about 30 mg to about 80 mg of cerdulatinib, or a pharmaceutically acceptable salt thereof, and at least one pharmaceutically acceptable excipient or carrier.

For example, the method can comprise administering to the patient a twice daily a dose of, or about, 5 mg, 10 mg, 15 mg, 20 mg, 25 mg. 30 mg, 35 mg, 40 mg, 45 mg, 50 mg, 55 mg, 60 mg, 65 mg, 70 mg, 75 mg, 80 mg, or more, or a number or a range between any two of these values, of the SYK or JAK inhibitor (e.g., cerdulatinib or a pharmaceutically acceptable salt thereof). In some embodiments, the patient is administered with a twice daily a dose of, or about, 35 mg or 40 mg of the SYK or JAK inhibitor (e.g., cerdulatinib or a pharmaceutically acceptable salt thereof).

In some embodiments, the administration of the SYK or JAK inhibitor (e.g., cerdulatinib, or a pharmaceutically acceptable salt thereof) is once daily. In some embodiments, the administration is twice daily. In some embodiments, the administration is three times daily.

In some embodiments, the therapeutically effective amount of the SYK or JAK inhibitor (e.g., cerdulatinib, or a pharmaceutically acceptable salt thereof) used in the methods provided herein is at least about 10 mg per day. For example, the therapeutically effective amount of the SYK or JAK inhibitor (e.g., cerdulatinib, or a pharmaceutically acceptable salt thereof) can be at least about 10, 20, 30, 40, or 50 mg per dosage. In one embodiment, the therapeutically effective amount of the SYK or JAK inhibitor (e.g., cerdulatinib, or a pharmaceutically acceptable salt thereof) is at least about 10, 20, 30, 40, 50, 60, 70, 80, 90 or 100 mg per day.

The specific amount of a SYK or JAK inhibitor (e.g., cerdulatinib) described herein refers to the amount of the inhibitor free base, such as the cerdulatinib free base, i.e., the compound of formula I. However, it is understood that a pharmaceutically acceptable salt, co-crystal or solvate of the SYK or JAK inhibitor or a mixture thereof may be administered in an amount that provides the stated amount of the SYK or JAK inhibitor. Examples of pharmaceutically acceptable salts of the SYK or JAK inhibitor include those derived from inorganic or organic acids, such as acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, edisylate, fumarate. lucoheptanoate, glycerophosphate, hemisulfate, heptanoate, hexanoate, hydrochloride, bis-hydrochloride, hydrobromide, hydroiodide, 2-hydroxyethanesulfonate, lactate, maleate, methanesulfonate, bis-methanesulfonate, 2-naphthalenesulfonate, naphthalene disulfate, nicotinate, oxalate, pamoate, pectinate, persulfate, 3-phenyl-propionate, picrate, pivalate, propionate, succinate, tartrate, thiocyanate, tosylate, undecanoate. hydrohalides (e.g., hydrochlorides and hydrobromides), sulfates, phosphates, nitrates, sulphamates, malonates, salicylates, methylene-bis-b-hydroxynaphthoates, gentisates, isethionates, di-ptoluoyltartrates, ethanesulphonates, cyclohexylsulphamates, quinates, and the like.

A SYK or JAK inhibitor of the present disclosure (e.g., cerdulatinib) or a salt thereof can be administered in unsolvated forms as well as solvated forms, including hydrated forms, or form co-crystals with another compound. “Hydrate” refers to a complex formed by combination of water molecules with molecules or ions of the solute. “Solvate” refers to a complex formed by combination of solvent molecules with molecules or ions of the solute. The solvent can be an organic compound, an inorganic compound, or a mixture of both. Solvate is meant to include hydrate, hemi-hydrate, channel hydrate etc. Some examples of solvents include, but are not limited to, methanol, N,N-dimethylformamide, tetrahydrofuran, dimethylsulfoxide, and water.

In some embodiments, the SYK or JAK inhibitor is cerdulatinib. Cerdulatinib can be administered as a hydrochloride salt (cerdulatinib HCl). In some embodiments, the cerdulatinib HCl is in a crystalline form. In some embodiments, the cerdulatinib HCl is in a crystalline form characterized by an X-ray powder diffractogram comprising peaks at 8.7, 15.9, and 20.0° 2θ, each ±0.2° 2θ, as determined on a diffractometer using Cu-Kα radiation (cerdulatinib HCl Form I). In some embodiments, cerdulatinib HCl Form I is further characterized by one or more peaks at 11.5, 22.5, and 25.5° 2θ, each ±0.2° 2θ. In some embodiments, cerdulatinib HCl Form I is further characterized by a differential scanning calorimetry curve comprising an endotherm with onset at about 288° C.

### 4. Combination Treatments

In one embodiment, the treatment methods can further include administration of an effective amount of another agent. In some embodiments, the SYK or JAK inhibitor (e.g., cerdulatinib) is co-administered with an effective amount of the another agent or a pharmaceutically acceptable salt, co-crystal or solvate thereof. In some embodiments, the agent is co-administered with the SYK or JAK inhibitor simultaneously or sequentially.

In some embodiments, the second agent is a corticosteroid. Non-limiting examples include methylprednisolone (in particular in patients with a rheumatic disease), dexamethasone (in particular in patients with FHLH).

In some embodiments, the second agent is a cytoablative therapy. Non-limiting examples include cyclophosphamide (in particular in patients with JIA and MAS), etoposide (in particular in patients with FHLH), rituximab (in particular in Epstein-Barr virus (EBV)-associated HLH), antithymocyte globulin (in particular for patients at bone marrow transplant phase of FHLH therapy), alemtuzumab (in particular in patients with FHLH or SLE-associated MAS).

In some embodiments, the second agent is a T-cell modulator. Non-limiting examples include calcineurin (e.g., cyclosporine) which prevents production of IL-2, and abatacept, which inhibits CD28 signaling of T cells.

In some embodiments, the second agent is a cytokine inhibitor, such inhibitors targeting INFγ, IL-1β, IL-18, IL-33, IL-6, and/or TNF.

In some embodiments, the second agent targets the underlying disease or condition, such as SARS-COV-2 infection. Non-limiting examples include lopinavir, ritonavir, oseltamivir (Tamiflu), favipiravir, fingolimod, methylprednisolone, bevacizumab, chloroquine phosphate, chloroquine, hydroxy chloroquine sulfate and remdesivir.

### 5. Pharmaceutical Compositions and Administration Routes

Some embodiments provided herein are directed to pharmaceutical compositions comprising an effective amount of the SYK or JAK inhibitor (e.g., cerdulatinib) and at least one pharmaceutically acceptable carrier or excipient.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” and the term “pharmaceutically acceptable excipient” refer to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the cancerous tissue or a tissue adjacent to the cancerous tissue.

As used herein, the term “pharmaceutically acceptable salt” refers to any acid or base addition salt whose counter-ions are non-toxic to the patient in pharmaceutical doses of the salts. A host of pharmaceutically acceptable salts are well known in the pharmaceutical field. If pharmaceutically acceptable salts of the compounds disclosed herein are utilized in these compositions, those salts are preferably derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate, undecanoate, hydrohalides (e.g., hydrochlorides and hydrobromides), sulphates, phosphates, nitrates, sulphamates, malonates, salicylates, methylene-bis-b-hydroxynaphthoates, gentisates, isethionates, di-p-toluoyltartrates, ethanesulphonates, cyclohexylsulphamates, quinates, and the like. Pharmaceutically acceptable base addition salts include, without limitation, those derived from alkali or alkaline earth metal bases or conventional organic bases, such as triethylamine, pyridine, piperidine, morpholine, N-methylmorpholine, ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

As used herein, the term “formulated” or “formulation” refers to the process in which different chemical substances, including one or more pharmaceutically active ingredients, are combined to produce a dosage form. In certain embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time, whereas an immediate release formulation is a formulation which is designed to quickly release a therapeutic agent in the body over a shortened period of time.

Carriers or excipients can be used to produce compositions. The carriers or excipients can be chosen to facilitate administration of the SYK or JAK inhibitor (e.g., cerdulatinib). Examples of carriers include calcium carbonate, calcium phosphate, various sugars such as lactose, glucose, or sucrose, or types of starch, cellulose derivatives, gelatin, vegetable oils, polyethylene glycols and physiologically compatible solvents. Examples of physiologically compatible solvents include sterile solutions of water for injection (WFI), saline solution, and dextrose.

Suitable dosage forms, in part, depend upon the use or the route of administration, for example, oral, transdermal, transmucosal, inhalant, or by injection (parenteral). Such dosage forms should allow the compound to reach target cells. Other factors are well known in the art, and include considerations such as toxicity and dosage forms that retard the compound or composition from exerting its effects. Techniques and formulations generally may be found in The Science and Practice of Pharmacy, 21st edition, Lippincott, Williams and Wilkins, Philadelphia, Pa., 2005 (hereby incorporated by reference herein).

The SYK or JAK inhibitor (e.g., cerdulatinib) can be administered by different routes including intravenous, intraperitoneal, subcutaneous, intramuscular, oral, transmucosal, rectal, transdermal, or inhalant. In some embodiments, the SYK or JAK inhibitor (e.g., cerdulatinib) can be administered by oral administration. For oral administration, for example, the SYK or JAK inhibitor (e.g., cerdulatinib) can be formulated into conventional oral dosage forms such as capsules, tablets, and liquid preparations such as syrups, elixirs, and concentrated drops.

For inhalants, the SYK or JAK inhibitor (e.g., cerdulatinib) may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. The SYK or JAK inhibitor (e.g., cerdulatinib) may also be used in combination with other inhaled therapies, for example corticosteroids such as fluticasone propionate, beclomethasone dipropionate, triamcinolone acetonide, budesonide, and mometasone furoate; beta agonists such as albuterol, salmeterol, and formoterol; anticholinergic agents such as ipratropium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

Pharmaceutical preparations for oral use can be obtained, for example, by combining the SYK or JAK inhibitor (e.g., cerdulatinib) with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol: cellulose preparations. for example, maize starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP, povidone). If desired, disintegrating agents may be added, such as the cross-linked polyvinylpyrrolidone, agar, or alginic acid, or a salt thereof such as sodium alginate.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the SYK or JAK inhibitor (e.g., cerdulatinib) may be dissolved or suspended in suitable liquids, such as fatty oils, liquid paraffin, or liquid polyethylene glycols (PEGs). In addition, stabilizers may be added.

Alternatively, injection (parenteral administration) may be used, e.g., intramuscular, intravenous, intraperitoneal, and/or subcutaneous. For injection, the SYK or JAK inhibitor (e.g., cerdulatinib) can be formulated in sterile liquid solutions, such as in physiologically compatible buffers or solutions, such as saline solution, Hank's solution, or Ringer's solution. In addition, the SYK or JAK inhibitor (e.g., cerdulatinib) may be formulated in solid form and redissolved or suspended immediately prior to use. Lyophilized forms can also be produced.

The disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

All publications, patent applications, patents, sequences, database entries (e.g., PUBMED, NCBI or PUBCHEM accession numbers), and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A method for treating or preventing cytokine release syndrome in a patient in need thereof, comprising administering to the patient an effective amount of a SYK inhibitor or a pharmaceutically acceptable salt thereof.

2. The method of claim 1, wherein the SYK inhibitor is a dual SYK/JAK inhibitor or a pharmaceutically acceptable salt thereof.

3. The method of claim 1 or 2, wherein SYK inhibitor is cerdulatinib or a pharmaceutically acceptable salt thereof.

4. The method of any preceding claim, wherein the patient has a viral infection.

5. The method of claim 4, wherein the viral infection is an infection by a coronavirus.

6. The method of claim 5, wherein the coronavirus is SARS-COV-2 or SARS-COV.

7. The method of any one of claims 1-4, wherein the viral infection is an infection by an influenza virus.

8. The method of claim 7, wherein the influenza virus is the H5N1 influenza A virus or the H7N9 influenza A virus.

9. The method of any one of claims 1-3, wherein the patient has been treated with an immunomodulating agent.

10. The method of claim 9, wherein the immunomodulating agent is selected the group consisting of an anti-CD3 antibody, an anti-CD52 antibody, and an anti-CD20 antibody.

11. The method of any one of claims 1-3, wherein the patient has been treated with T-cells modified with chimeric antigen receptors (CAR-T).

12. The method of any one of claims 1-3, wherein the patient suffers from a disease or condition selected from the group consisting of familial hemophagocytic lymphohistiocytosis (FHLH), systemic juvenile idiopathic arthritis (JIA), adult-onset Still's disease (AOSD), macrophage activation syndrome (MAS) and sepsis.

13. The method of any one of claims 1-3, wherein the patient suffers from a disease or condition selected from the group consisting of graft-versus-host disease (GVHD), acute respiratory distress syndrome (ARDS), sepsis, Ebola, avian influenza, smallpox, and systemic inflammatory response syndrome (SIRS).

14. The method of any one of claims 1-3, wherein the patient has had a stem cell transplant.

15. The method of any preceding claim, further comprising administering to the patient an effective amount of a second agent selected from the group consisting of a corticosteroid, a cytoablative agent, a T-cell modulator, and a cytokine inhibitor, or a pharmaceutically acceptable salt thereof.

16. The method of claim 15, wherein the second agent is selected from the group consisting of methylprednisolone, dexamethasone, cyclophosphamide, etoposide, rituximab, antithymocyte globulin, alemtuzumab, cyclosporine, abatacept, and inhibitors of INFγ, IL-1β,IL-18, IL-33, IL-6, and TNF.

17. The method of any preceding claim, further comprising administering to the patient an effective amount of a drug selected from the group consisting lopinavir, ritonavir, oseltamivir, favipiravir, fingolimod, methylprednisolone, bevacizumab, chloroquine phosphate, chloroquine, hydroxychloroquine sulfate, remdesivir, and combinations thereof.

18. A SYK inhibitor, e.g., cerdulatinib or a pharmaceutically acceptable salt thereof, for the treatment or prevention of cytokine release syndrome (CRS) in a subject, e.g., CRS in a human patient due to a viral infection; especially, treatment of CRS in a human patient caused by a coronavirus (e.g., SARS-COV-2 or SARS-COV) or an influenza virus.

19. Use of a SYK inhibitor, e.g., cerdulatinib or a pharmaceutically acceptable salt thereof, for the treatment or prevention of cytokine release syndrome (CRS) in a subject, e.g., CRS in a human patient due to a viral infection; especially, treatment of CRS caused by a coronavirus (e.g., SARS-COV-2 or SARS-COV) or an influenza virus.
